(19) United States
(12) Patent Application Publication
(10) Pub. No.: US 2026/0131165 A1
(43) Pub. Date: May 14, 2026

(54) INTRA-FRACTIONAL PATIENT POSITION VERIFICATION

(71) Applicant: VARIAN MEDICAL SYSTEMS, INC., Palo Alto, CA (US)

(72) Inventors: Daniel MORF, Buch am Irchel (CH); Stephen THOMPSON, Marina, CA (US)

(73) Assignee: VARIAN MEDICAL SYSTEMS, INC., Palo Alto, CA (US)

(21) Appl. No.: 18/945,529

(22) Filed: Nov. 13, 2024

(51) Int. Cl. A61N 5/10

(52) U.S. Cl. CPC A61N 5/107; A61N 5/1049; A61N 5/1081; A61N 2005/1061; A61N 2005/1087; A61N 2005/1089

(57) ABSTRACT

A radiation treatment system includes a rotatable gantry that is configured to rotate about a treatment isocenter of the radiation therapy system, a treatment-delivering radiation source that is coupled to the rotatable gantry and directs a treatment beam through the treatment isocenter of the radiation therapy system, a first X-ray imager that is coupled to the rotatable gantry, and a second X-ray imager that is mounted on a component of the radiation therapy system and is fixed in position relative to a couch of the radiation therapy system while the rotatable gantry rotates about the treatment isocenter.

### BACKGROUND

Unless otherwise indicated herein, the approaches described in this section are not prior art to the claims in this application and are not admitted to be prior art by inclusion in this section.

Radiation therapy (also called radiotherapy) is a cancer treatment that employs high doses of ionizing radiation, such as X-rays or high-energy electrons, protons, or other heavy charged particles, to kill cancer cells. Generally, radiation therapy is a localized treatment for a specific target tissue, such as a cancerous tumor. Ideally, radiation therapy is performed on a planning target volume (i.e., the target tissue) that spares the surrounding normal tissue from receiving doses above specified tolerances, thereby minimizing risk of damage to healthy tissue. For example, to accurately supply a planned radiation dose, the spatial distribution of delivered radiation dose within the patient must closely match the spatial distribution of the planned radiation dose. So that the planned radiation dose is correctly supplied to the planning target volume during radiation therapy, the patient should be correctly positioned relative to the radiation source that provides the radiation therapy. In addition, precisely controlling the position of the radiation source relative to the patient is a significant factor in accurately targeting tissue in the patient.

### SUMMARY

According to various embodiments, a radiation therapy system includes a static X-ray imaging system that is fixed in position relative to a couch of the radiation therapy system and enables position verification of a patient during treatment. In the embodiments, after a patient is correctly positioned on the couch for a treatment fraction, each imager of the static X-ray imaging system acquires a reference X-ray image of a region of patient anatomy. During treatment, subsequent position verification X-ray images are acquired by the imagers of the static X-ray imaging system. Patient motion and/or internal anatomy motion is detected by comparing a reference X-ray image for a particular imager of the static X-ray imaging system to the most recent position verification X-ray image acquired by that particular imager. Because each imager of the static X-ray imaging system is fixed in position relative to the couch, X-ray images acquired by the static X-ray imaging system are taken in a patient coordinate system rather than a rotating system coordinate system. Consequently, motion of the patient and/or internal anatomy can be detected by directly comparing a position verification X-ray image acquired by an imager of the static X-ray system to the reference X-ray image acquired by that imager. As a result, motion detection can be performed quickly and reliably throughout a treatment fraction. Advantageously, this efficient process can be employed even when the couch is moved to multiple positions during the treatment fraction. Additionally or alternatively, in embodiments in which the static X-ray system includes multiple imagers, a reference set of three-dimensional coordinate values of a feature disposed within the region of patient anatomy can be determined based on multiple reference X-ray images (one for each imager of the static X-ray system) and a position verification set of three-dimensional coordinate values of the feature can be determined based on multiple position verification X-ray images (one for each imager of the static X-ray system). In such embodiments, patient motion and/or internal anatomy motion is detected by comparing the reference set of three-dimensional coordinate values to the most recent position verification set of three-dimensional coordinate values that has been determined.

According to some embodiments, a radiation treatment system includes: a rotatable gantry that is configured to rotate about a treatment isocenter of the radiation therapy system; a treatment-delivering radiation source that is coupled to the rotatable gantry and directs a treatment beam through the treatment isocenter of the radiation therapy system; a first X-ray imager that is coupled to the rotatable gantry; and a second X-ray imager that is mounted on a component of the radiation therapy system and is fixed in position relative to a couch of the radiation therapy system while the rotatable gantry rotates about the treatment isocenter.

According to some embodiments, a computer-implemented method of performing a radiation therapy treatment fraction on a target volume within an anatomical region of a patient includes: while the patient is disposed on a couch of a radiation therapy system, acquiring multiple X-ray images of the anatomical region with a first X-ray imager that is coupled to a rotatable gantry of the radiation therapy system; reconstructing a digital volume of the region based on the multiple X-ray images of the anatomical region; adjusting a position of the couch based on the digital volume; after adjusting the position of the couch, acquiring at least one reference X-ray image of the anatomical region with a second X-ray imager that is mounted on a component of the radiation therapy system and is fixed in position relative to the couch while the rotatable gantry rotates about a treatment isocenter of the radiation therapy system; after acquiring the at least one reference X-ray image, beginning the treatment fraction; during the treatment fraction, acquiring at least one position verification X-ray image of the anatomical region with the second X-ray imager; and determining a motion threshold is exceeded based on the reference X-ray image and the position verification X-ray image.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are, therefore, not to be considered limiting of its scope. The disclosure will be described with additional specificity and detail through use of the accompanying drawings.

FIG. 1 is a perspective view of a radiation therapy system that can beneficially implement various embodiments.

FIG. 2 schematically illustrates a side view of the radiation therapy system of FIG. 1, according to various embodiments.

FIG. 3 schematically illustrates a digital volume that is constructed based on projection images generated by one or more X-ray images included in the radiation therapy system of FIG. 1, according to various embodiments.

FIG. 4 is a schematic end view of a static X-ray imaging system and a couch, according to various embodiments.

FIG. 5 is a perspective view of a radiation therapy system, according to various embodiments.

FIG. 6 schematically illustrates a base stand and a gantry of the radiation therapy system of FIG. 5, according to various embodiments.

FIG. 7 is a schematic plan view of a static X-ray imaging system with repositionable imagers and a couch, according to various embodiments.

FIG. 8 is a flowchart illustrating the steps of a computer-implemented method of performing a radiation therapy treatment fraction on a target volume within an anatomical region of a patient, according to various embodiments.

FIG. 9 is an illustration of a computing device configured to perform various embodiments of the present disclosure.

FIG. 10 is a block diagram of an illustrative embodiment of a computer program product 1000 for implementing a method for performing a radiation therapy treatment fraction on a target volume within an anatomical region of a patient, according to various embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the disclosure, as generally described herein, and illustrated in the figures, can be arranged, substituted, combined, and designed in a wide variety of different configurations, all of which are explicitly contemplated and make part of this disclosure. Although the terms “first,” “second,” and “third” are used to describe various elements, these elements should not be limited by these terms. These terms are used to distinguish one element from another. For example, a first element may be referred to as a second element, and vice versa. Independent of the grammatical term usage, individuals with male, female or other gender identities are included within the term.

### INTRODUCTION

As noted previously, to accurately supply a planned radiation dose to a planning target volume during radiation therapy, the patient should be precisely positioned relative to the radiation source that provides the radiation therapy. For many planned treatments, it is also beneficial to monitor patient position throughout the time that radiation is delivered to the target volume. For example, in hypofractionated radiation therapy, more doses of radiation are delivered per treatment, so a patient can complete a course of radiation therapy much faster than conventional treatment. Hypofractionated radiation therapy allows patients to complete a planned treatment more quickly and can have fewer short-term side effects. However, hypofractionated radiation therapy generally involves prolonged beam-times (e.g., on the order of multiple minutes) that can increase the risk of organ or patient movement. Further, because the planned dose may be delivered in a small number of fractions (e.g., 1-5), mistreatment caused by patient and/or internal anatomy movement can be severe and difficult to compensate for in subsequent fractions, if any. As a result, various techniques have been developed for monitoring patient and/or internal anatomy intra-fraction motion, i.e., motion during the time that radiation is delivered to a target volume.

In some instances, surrogate monitoring technologies are employed to infer intra-fraction motion of the patient and/or internal anatomy, for example by optically monitoring a surface of the body of the patient. However, optical monitoring technologies cannot directly monitor internal anatomy, where significant motion oftentimes occurs (e.g., due to sporadic prostate movement). Because there can oftentimes be no reliable correlation between internal anatomy motion and the surface being monitored, optical surface monitoring is often unable to detect certain internal anatomy motion, making such monitoring unsuitable for hypofractionated radiation therapy. A further drawback of optical monitor technologies is that no blankets or clothes can be on the surface area being monitored, which can be very uncomfortable for a patient, particularly for a longer-duration treatment fraction.

In some instances, on-board imaging of the radiation therapy system itself can be used to detect intrafraction motion of the patient and/or internal anatomy. For example, the imager or imagers that are mounted on the rotating gantry with the treatment-delivering linear accelerator (LINAC) can be employed to acquire multiple radiographs of patient anatomy during treatment. However, these radiographs have a limited ability to enable detection of internal anatomy motion in three dimensions for multiple reasons. First, as two-dimensional snapshot images of patient anatomy, these during-treatment radiographs provide no three-dimensional 3D position information. Second, each such radiograph is taken from a different angle across the treatment arc, which makes feature detection or matching much more challenging and less reliable. Third, due to the 90 degree angular offset typically present between on-board imagers and the treatment beam, one of the two dosimetrically important axes is always missing in such radiographs, further compromising the ability of such radiographs to indicate motion of internal anatomy relative to the treatment beam. Fourth, many radiographs acquired by on-board imaging have limited utility for motion detection, since they are acquired from a point of view in which critical anatomical features are partially or completely blocked by high-density anatomy, such as bony structures.

In some instances, X-ray imaging systems external to a radiation therapy system have been employed to detect intrafraction motion of the patient and/or internal anatomy. Generally, such systems are installed in the room surrounding a radiation therapy system, for example in the ceiling and floor of the room. While external X-ray imaging systems can provide X-ray images of patient internal anatomy during treatment, there are significant drawbacks. First, such systems require significant modification to the facility containing the radiation therapy system, which can add cost and complexity to the installation of the associated radiation therapy system. Second, such external X-ray imaging systems inherently have a small field of view, due to the magnification caused by the distances of the X-ray source and imager from the patient anatomy being monitored. As a result, the region of patient anatomy that such imaging systems can monitor is limited, which can prevent timely detection of internal anatomy motion.

Accordingly, there is a need in the art for improved systems for monitoring the motion of internal anatomy of a patient during radiation therapy.

### Position Verification for C-Arm Gantry System

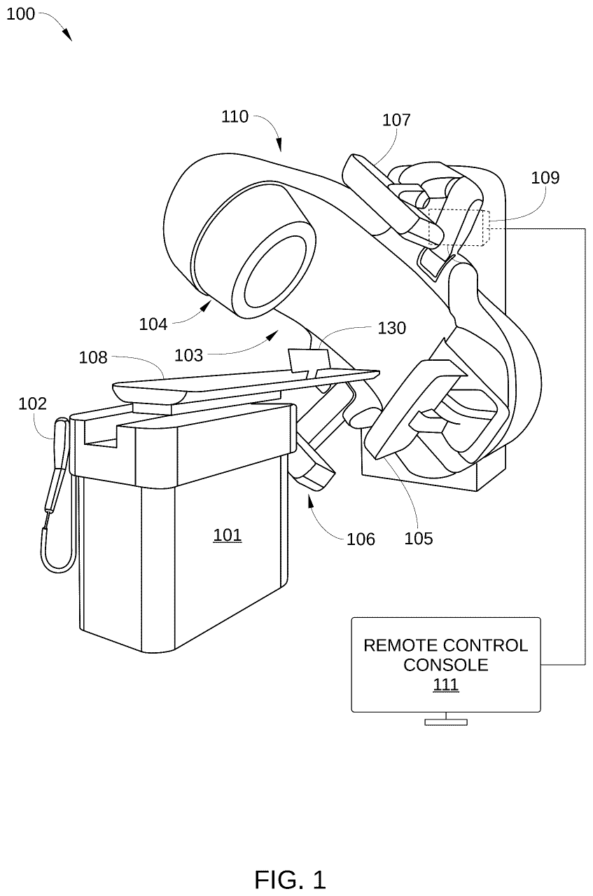

FIG. 1 is a perspective view of a radiation therapy system 100 that can beneficially implement various embodiments. Radiation therapy (RT) system 100 is a radiation system that may be configured to detect intra-fraction motion in near-real time using either optical or X-ray imaging techniques, or both. Thus, in some embodiments, RT system 100 is configured to provide stereotactic radiosurgery and precision radiotherapy for lesions, tumors, and conditions anywhere in the body where radiation treatment is indicated. As such, RT system 100 can include one or more of a linear accelerator (LINAC) 104 that generates an MV treatment beam of high energy X-rays or other radiation, one or more kilovolt (kV) imaging X-ray sources 106, one or more imaging panels 107 (e.g., an X-ray imager), a mega-Volt (MV) electronic portal imaging device (EPID) 105, and a static X-ray imaging system 130. In the embodiment illustrated in FIG. 1, RT system 100 is configured with a C-arm gantry 110 capable of infinite rotation via a slip ring connection.

In some embodiments, RT system 100 is capable of X-ray imaging of a target volume immediately prior to and/or during application of an MV treatment beam, so that an image-guided radiation therapy (IGRT) and/or an intensity-modulated radiation therapy (IMRT) process can be performed using X-ray imaging. For example, in some embodiments, such processes can include kV imaging of the target volume in conjunction with imaging generated by the MV treatment beam. RT system 100 may include one or more touchscreens (not shown) for patient information verification, couch motion controls 102, a radiation area 103, a couch positioning assembly 101, a couch 108 disposed on couch positioning assembly 101, and an image acquisition and treatment control computer 109, all of which are disposed within a treatment room. RT system 100 further includes a remote control console 111, which is disposed outside the treatment room and enables treatment delivery and patient monitoring from a remote location. Couch positioning assembly 101 is configured to precisely position couch 108 with respect to radiation area 103, including rotating couch 108 about an isocenter of RT system 100. In some embodiments, a center of rotation (not shown) of couch positioning assembly 101 is vertically aligned with the treatment isocenter. Motion controls 102 include input devices, such as buttons and/or switches, that enable a user to operate couch positioning assembly 101 to automatically and precisely position couch 108 to a predetermined location with respect to radiation area 103. Motion controls 102 also enable a user to manually position couch 108 to a particular location, such as a planned treatment position for a patient or anatomical target.

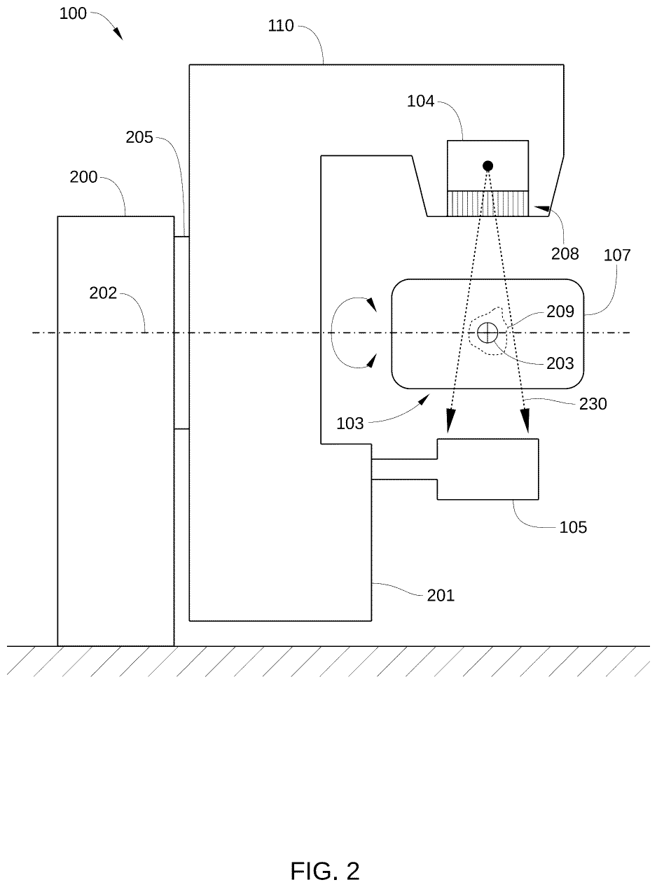

FIG. 2 schematically illustrates a side view of RT system 100, according to various embodiments. As shown, RT system 100 includes a base stand 200 and C-arm gantry 110. In FIG. 2, couch positioning assembly 101, couch 108, and imaging X-ray source 106 are omitted for clarity. Base stand 200 is a fixed support structure for components of RT treatment system 100, including C-arm gantry 110 and a drive system (not shown) for rotatably moving C-arm gantry 110 about a horizontal rotation axis 202. Base stand 200 rests on and/or is fixed to a support surface that is external to RT treatment system 100, such as a floor of an RT treatment facility. C-arm gantry 110 is rotationally coupled to base stand 200, for example via a bearing 205 (cross-hatched). C-arm gantry 110 is a support structure on which various components of RT system 100 are mounted, including LINAC 104, EPID 105, imaging X-ray source 106 (not shown in FIG. 2 for clarity), and imaging panel 107.

In the embodiment illustrated in FIGS. 1 and 2, imaging panel 107 is depicted as a planar device, whereas in other embodiments, imaging panel 107 can have a curved configuration. In the embodiment illustrated in FIGS. 1 and 2, RT system 100 includes a single imaging panel and a single corresponding imaging radiation source in addition to EPID 105. In other embodiments, RT system 100 can include two or more imaging panels, each with a corresponding imaging radiation source. Further, in some embodiments, couch positioning assembly 101 is configured to rotate, pitch, roll, and/or translate couch 108 relative to an isocenter 203 in order to move a patient to a treatment position. Isocenter 203 can be a treatment isocenter of RT system 100, which is a point in space about which various components mounted on C-arm gantry 110 rotate.

LINAC 104 is a radiation source, and typically includes one or more of an electron gun for generating electrons, an accelerating waveguide, an electron beam target, an electron beam transport means (such as a bending magnet) for directing the electron beam to the electron beam target, and/or a collimator assembly 208 for collimating and shaping a treatment beam 230 that originates from the electron beam target. Collimator assembly 208 typically includes one or more of a primary collimator that defines the largest available circular radiation field for treatment beam 230, a secondary collimator for providing a rectangular or square radiation field at isocenter 203 (for example via X-jaws and Y-jaws), and/or a multileaf collimator (MLC) for conforming treatment beam 230 to a PTV or other anatomical target. In other embodiments, LINAC 104 can be any other radiation source suitable for radiation therapy.

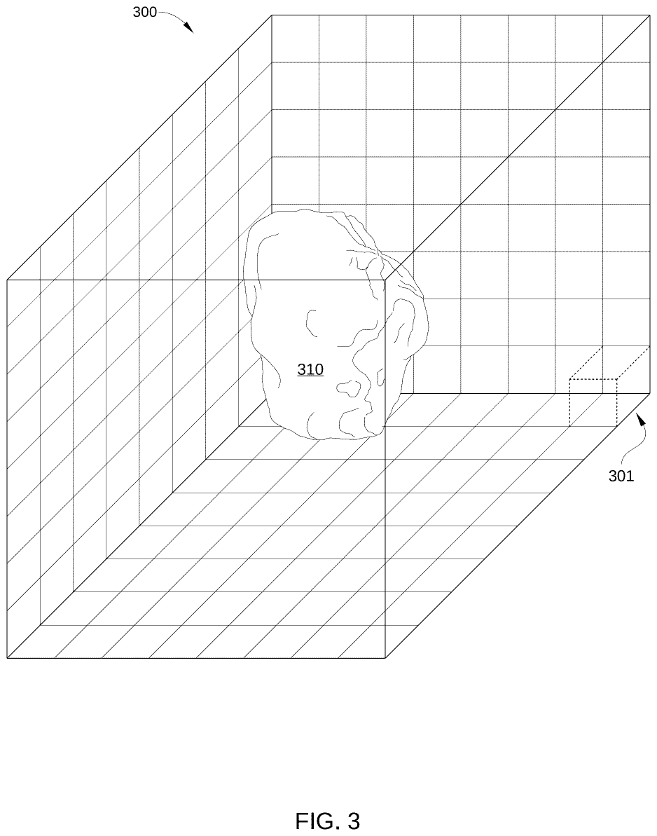

During radiation treatment, LINAC 104 is configured to generate treatment beam 230, which can include high-energy radiation (for example MV X-rays or MV electrons). In other embodiments, treatment beam 230 includes electrons, protons, and/or other heavy charged particles, ultra-high dose rate X-rays (e.g., for FLASH radiotherapy), and/or microbeams for microbeam radiation therapy. In addition, imaging panel 107 is configured to receive imaging radiation and generate suitable projection images therefrom. Further, in some embodiments, as treatment beam 230 is directed to isocenter 203 while C-arm gantry 110 rotates through a treatment arc, image acquisitions can be performed via EPID 105 to generate image data for target volume 209. For example, in such embodiments, EPID 105 generates one or more projection images of target volume 209 and/or a region of patient anatomy surrounding target volume 209. Thus, projection images (e.g., 2D X-ray images) of target volume 209 can be generated during portions of an IGRT or IMRT process via imaging panel 107 and/or EPID 105. Such projection images can then be employed to reconstruct a digital volume that corresponds to a three-dimensional (3D) region that includes target volume 209. That is, a 3D image of such a 3D region is reconstructed from the projection images. In some embodiments, cone-beam computed tomography (CBCT) and/or digital tomosynthesis (DTS) can be used to process the projection images generated by imaging panel 107 and generate a digital volume of the region including target volume 209. One embodiment of such a digital volume is described below in conjunction with FIG. 3.

FIG. 3 schematically illustrates a digital volume 300 that is reconstructed based on a plurality of CT views (e.g., several hundred) acquired by imaging panel 107 and/or EPID 105, according to various embodiments. Digital volume 300 includes a plurality of voxels 301 (dashed lines) of anatomical image data, where each voxel 301 corresponds to a different location within digital volume 300. For clarity, only a single voxel 301 is shown in FIG. 3. Digital volume 300 corresponds to a 3D region that includes target volume 310. In FIG. 3, digital volume 300 is depicted as an 8×8×8 voxel cube, but in practice, digital volume 300 generally includes many more voxels, for example one or more orders of magnitude more than are shown in FIG. 3. Once reconstructed from a set of CT views, cross-sectional images of digital volume 300 can be generated at any location an in any of various planes, including the axial plane (which passes through patient anatomy from anterior to posterior, dividing the anatomy into superior and inferior sections), the coronal plane (which passes through patient anatomy from left to right and divides the anatomy into anterior and posterior sections, the sagittal plane (which passes through patient anatomy from anterior to posterior and divides the anatomy into left and right sections), and/or the oblique plane (which passes through patient anatomy at an angle to the axial, coronal, and/or sagittal planes).

For purposes of discussion, target volume 310 can refer to the gross tumor volume (GTV), clinical target volume (CTV), or the planning target volume (PTV) for a particular treatment. The GTV depicts the position and extent of the gross tumor, for example what can be seen or imaged; the CTV includes the GTV and an additional margin for sub-clinical disease spread, which is generally not imageable; and the PTV is a geometric concept designed to ensure that a suitable radiotherapy dose is actually delivered to the CTV without adversely affecting nearby organs at risk. Thus, the PTV is generally larger than the CTV, but in some situations can also be reduced in some portions to provide a safety margin around an organ at risk. The PTV is typically determined based on imaging performed prior to the time of treatment, and alignment of the PTV with the current position of patient anatomy at the time of treatment is facilitated by X-ray imaging of digital volume 300.

Returning to FIGS. 1 and 2, during operation of RT treatment system 100, C-arm gantry 110 rotates about radiation area 103 when actuated by the drive system for rotatably moving C-arm gantry 110 about horizontal rotation axis 202. Imaging X-ray source 106 is configured to direct a conical beam of X-rays, referred to herein as imaging X-rays (not shown in FIG. 2 for clarity), through an isocenter 203 of RT system 100 to imaging panel 107. Ideally, isocenter 203 corresponds to the location of a target volume 209 to be treated, such as a PTV, a GTV, a CTV, and/or an internal target volume (ITV), among others.

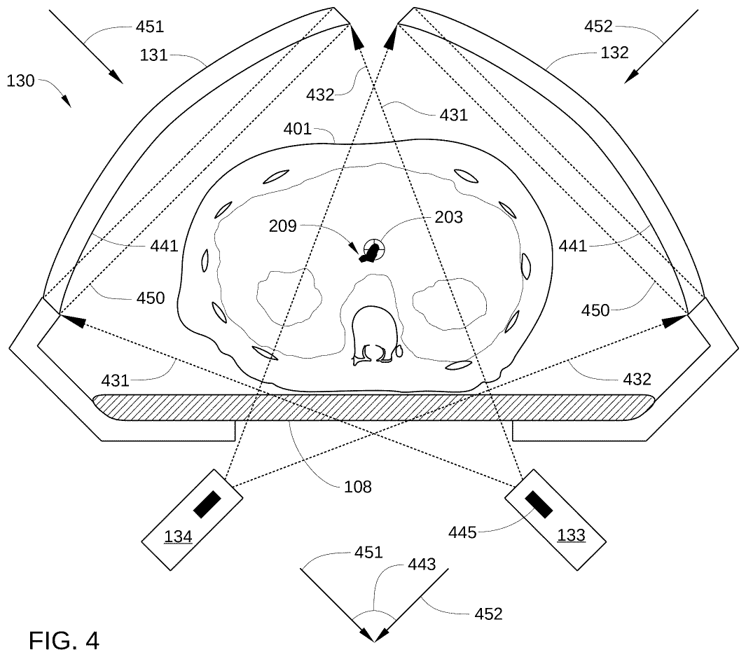

As shown in FIG. 1, RT system 100 includes static X-ray imaging system 130, which enables position verification during treatment by RT system 100. In the embodiment illustrated in FIG. 1, static X-ray imaging system 130 is mounted on a component of RT system 100 and is fixed in position relative to couch 108 while C-arm gantry 110 rotates about isocenter 203. As a result, X-ray images acquired by static X-ray imaging system 130 are taken in a patient coordinate system rather than a rotating system coordinate system associated with C-arm gantry 110. Thus, unlike rotating imagers of RT system 100, such as imaging panel 107 and/or EPID 105, an imager of static X-ray imaging system 130 maintains an identical field of view of a particular region of patient anatomy throughout a treatment fraction. Consequently, an image acquired by an imager of static X-ray imaging system 130 can be directly compared to a reference image acquired by that imager to detect motion. By contrast, a rotating imager of RT system 100 views a region of patient anatomy from constantly changing angles during a treatment fraction, which greatly complicates the analysis involved in feature matching and motion detection. Further, in embodiments in which static X-ray imaging system 130 includes multiple X-ray imagers, a reference set of three-dimensional coordinate values of a particular feature disposed within the region of patient anatomy can be determined based on multiple reference X-ray images (one for each imager of the static X-ray system) and a position verification set of three-dimensional coordinate values of the feature disposed can be determined based on multiple position verification X-ray images (one for each imager of the static X-ray system). For example, the particular feature can be a high-contrast feature disposed within the anatomical region, such as a fiducial marker, a portion of boney anatomy, and/or the like. Thus, each set of three-dimensional coordinate values can be a set of coordinates in (three dimensions) of the particular feature. In such embodiments, patient motion and/or internal anatomy motion is detected by comparing the reference set of three-dimensional coordinate values of the feature to the most recent position verification set of three-dimensional coordinate values that has been determined. Various embodiments of static X-ray imaging system 130 are described below in conjunction with FIG. 4.

FIG. 4 is a schematic end view of a static X-ray imaging system 130 and couch 108 (cross-hatched), according to various embodiment. Static X-ray imaging system 130 includes one or more imagers, each with a corresponding X-ray source. In the embodiment illustrated in FIG. 4, static X-ray imaging system 130 includes a first X-ray imager 131, a second X-ray imager 132, a first X-ray source 133, and a second X-ray source 134, all of which are coupled to or mounted on couch 108.

As shown, first X-ray source 133 directs imaging X-rays 431 through a region of patient anatomy 401 to first X-ray imager 131 and second X-ray source 134 directs imaging X-rays 432 through the region of patient anatomy 401 to second X-ray imager 132. In addition, first X-ray imager 131, second X-ray imager 132, first X-ray source 133, and second X-ray source 134 are coupled to couch 108. Consequently, when couch 108 is moved to multiple locations during a particular treatment fraction, the components of static X-ray imaging system 130 maintain the same relative orientation to couch 108 and patient anatomy 401. For example, in stereotactic body radiotherapy (SBRT), multiple treatment beams may be carefully targeted at target volume 209 during a single treatment fraction, where each treatment beam is delivered at a different preferential delivery angle. In some instances, to achieve each delivery angle, couch 108 is rotated about isocenter 203. According to various embodiments, static X-ray imaging system 130 maintains the same relative orientation to couch 108 and patient anatomy 401 throughout the treatment fraction, which greatly facilitates motion detection.

In the embodiment illustrated in FIG. 4, static X-ray imaging system 130 includes multiple X-ray imagers, each with an associated X-ray source. Thus, in such embodiments, static X-ray imaging system 130 can operate as a stereoscopic imaging system that can simultaneously acquire multiple X-ray images of patient anatomy 401. As shown, first X-ray imager 131 is oriented with a first viewing direction 451 toward couch 108 and second X-ray imager 132 is oriented with a second viewing direction 452 toward couch 108. In some embodiments, to facilitate motion detection, an imaging separation angle 443 between first viewing direction 451 and second viewing direction 452 is 90 degrees. In other embodiments, for example due to physical constraints on the size and location of static X-ray imaging system 130, imaging separation angle 443 is less than or greater than 90 degrees. In alternative embodiments, static X-ray imaging system 130 includes a single X-ray imager and a single corresponding X-ray source.

First X-ray imager 131 and/or second X-ray imager 132 can be any technically feasible digital radiography device, such as a solid-state X-ray digital flat-panel detector. Thus, in some embodiments, first X-ray imager 131 and/or second X-ray imager 132 can be implemented as an indirect detector containing a layer of scintillator material, and in other embodiments, first X-ray imager 131 and/or second X-ray imager 132 can be implemented as a direct conversion imager that employs photoconductors. In the embodiment illustrated in FIG. 4, first X-ray imager 131 and second X-ray imager 132 are configured with a curved detector panel 440. In such embodiments, curved detector panel 440 can provide increased space for a patient disposed on couch 108 without being positioned farther away from couch 108. In such embodiments, curved detector panel 440 includes a curved X-ray-receiving surface 441. In alternative embodiments, first X-ray imager 131 and/or second X-ray imager 132 can be configured with a flat detector panel 450 (dashed lines).

First X-ray source 133 and/or second X-ray source 134 can be any technically feasible X-ray-generating device, such as an X-ray tube. Compared to on-board imagers of RT system 100 and imagers mounted within the treatment room, first X-ray imager 131, second X-ray imager 132, first X-ray source 133, and second X-ray source 134 are positioned in close proximity to patient anatomy 401. Consequently, the power needed for first X-ray source 133 and second X-ray source 134 to generate sufficient X-rays for image acquisition is significantly less than that needed by X-ray source 106. As a result, in some embodiments, first X-ray source 133 and second X-ray source 134 can each be configured with a fixed anode 445 rather than a larger rotating anode more suitable for higher power applications.

### Position Verification for O-Ring Gantry System

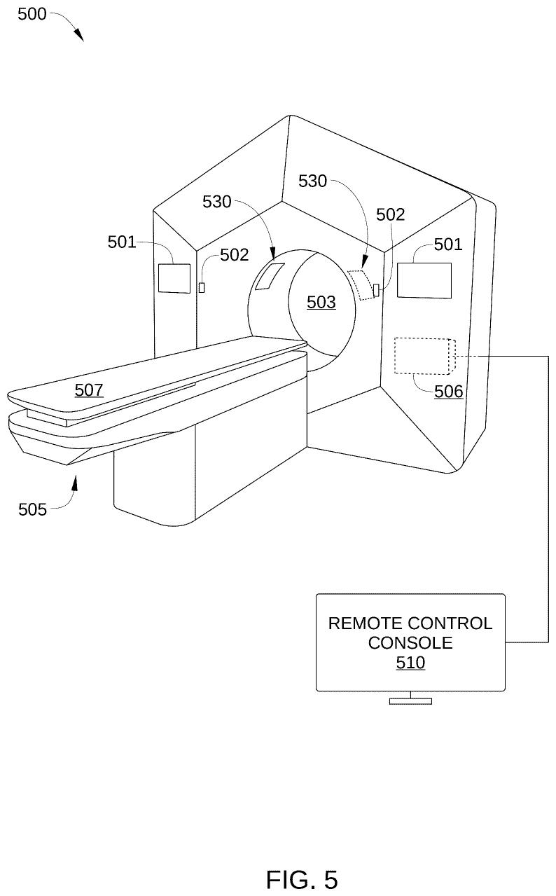

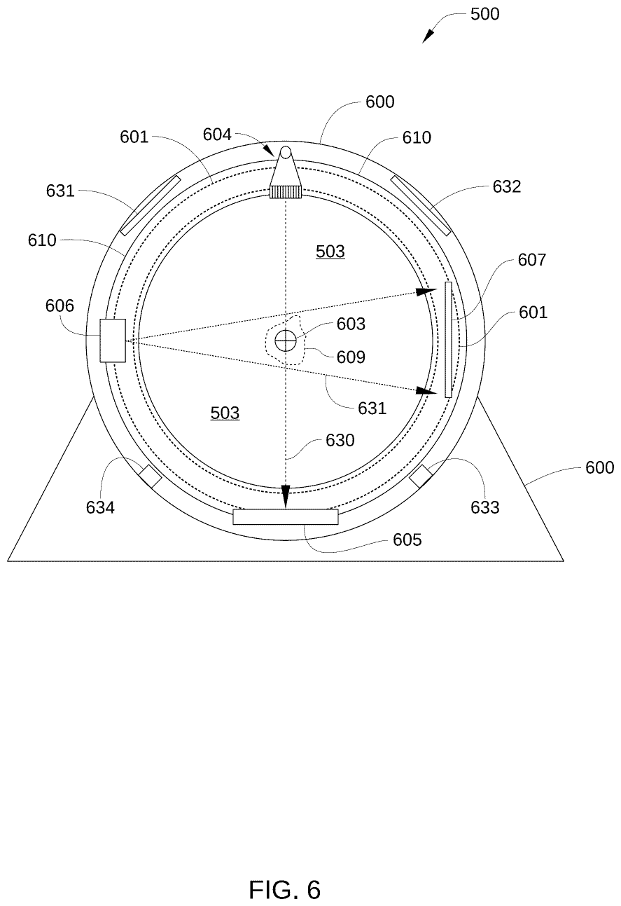

In the embodiment illustrated in FIGS. 1 and 2, C-arm gantry 110 of RT system 100 is capable of infinite rotation via a slip ring connection. In other embodiments, a radiation therapy system can be configured with a ring-based gantry that is positioned about an isocenter of the radiation therapy system. One such embodiment is described below in conjunction with FIGS. 5 and 6.

FIG. 5 is a perspective view of an RT system 500, according to various embodiments. In some embodiments, RT system 500 can be consistent with RT system 100 of FIGS. 1 and 2, except that RT system 500 is configured with a circular or ring-based gantry. As such, RT system 500 can include one or more touchscreens 501, couch motion controls 502, a bore 503, a base positioning assembly 505, a couch 507 disposed on base positioning assembly 505, an image acquisition and treatment control computer 506, and a static X-ray imaging system 530, all of which are disposed within a treatment room. RT system 500 further includes a remote control console 510, which is disposed outside the treatment room and enables treatment delivery and patient monitoring from a remote location. Base positioning assembly 505 is configured to precisely position couch 507 with respect to bore 503, and motion controls 502 include input devices, such as button and/or switches, that enable a user to operate base positioning assembly 505 to automatically and precisely position couch 507 to a predetermined location with respect to bore 503. Motion controls 502 also enable a user to manually position couch 507 to a predetermined location. Static X-ray imaging system 530 can be consistent with static X-ray imaging system 130 of FIGS. 1 and 2.

FIG. 6 schematically illustrates a base stand 600 and gantry 610 of RT system 500, according to various embodiments. Covers, base positioning assembly 505, couch 507, and other components of RT system 500 are omitted in FIG. 6 for clarity. Base stand 600 is a fixed support structure for components of RT system 500, including gantry 610 and a drive system 601 (dashed lines) for rotatably moving gantry 610. Base stand 600 rests on and/or is fixed to a support surface that is external to RT system 500, such as a floor of a radiotherapy treatment facility. Gantry 610 is rotationally coupled to base stand 600 and is a support structure on which various components of RT system 500 are mounted, including a LINAC 604, an EPID 605, an imaging X-ray source 606, and an X-ray imager 607. As shown, components of static X-ray imaging system 530 are mounted on or coupled to one or more stationary portions of RT system 500, such as base stand 600. Thus, when gantry 610 rotates about isocenter 603 of RT system 500, the components of static X-ray imaging system 530 remain stationary relative to couch 507.

During operation of RT system 500, drive system 601 rotationally actuates gantry 610, so that gantry 610 rotates about bore 503. LINAC 604 generates an MV treatment beam 630 of high energy X-rays (or in some embodiments electrons, protons, and/or other heavy charged particles, ultra-high dose rate X-rays (e.g., for FLASH radiotherapy) or microbeams for microbeam radiation therapy) and EPID 605 is configured to acquire X-ray images with treatment beam 630. Imaging X-ray source 606 is configured to direct a conical beam of X-rays, referred to herein as imaging X-rays 631, through an isocenter 603 of RT system 500 to X-ray imager 607, and isocenter 603 typically corresponds to the location of a target volume 609 to be treated. In the embodiment illustrated in FIG. 6, X-ray imager 607 is depicted as a planar device, whereas in other embodiments, X-ray imager 607 can have a curved configuration. In the embodiment illustrated in FIG. 6, RT system 500 includes a single X-ray imager and a single corresponding imaging X-ray source. In other embodiments, RT system 500 can include two or more on-board X-ray imagers, each with a corresponding imaging X-ray source.

According to various embodiments, the RT system 500 includes static X-ray imaging system 530, which enables position verification during treatment by RT system 500. As noted above, static X-ray imaging system 530 is mounted on one or more stationary components of RT system 500, such as base stand 600, and is fixed in position relative to couch 507 while gantry 610 rotates about isocenter 603. As a result, X-ray images acquired by static X-ray imaging system 530 are taken in a patient coordinate system rather than a rotating system coordinate system associated with gantry 610. Thus, unlike rotating imagers of RT system 500, such as EPID 605 and/or X-ray imager 607, an imager of static X-ray imaging system 530 maintains an identical field of view of a particular region of patient anatomy throughout a treatment fraction.

Static X-ray imaging system 530 includes one or more imagers, each with a corresponding X-ray source. In the embodiment illustrated in FIG. 6, static X-ray imaging system 530 includes a first X-ray imager 631, a second X-ray imager 632, a first X-ray source 633, and a second X-ray source 634, all of which are coupled to or mounted on base stand 600. In operation, first X-ray source 633 directs imaging X-rays (not shown) through target volume 609 to first X-ray imager 631 and second X-ray source 634 directs imaging X-rays (not shown) through target volume 609 to second X-ray imager 632.

In the embodiments illustrated in FIGS. 5 and 6, components of static X-ray imaging system 530 are mounted on drive stand 600 or another stationary component of RT system 500. In other embodiments, some or all components of static X-ray imaging system 530 can be mounted on couch 507.

### Repositionable Imagers in a Static Position Verification Imaging System

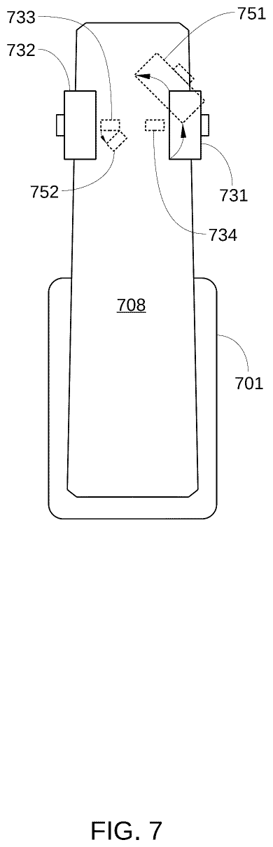

In some embodiments, a static X-ray imaging system, such as static X-ray imaging system 130 of FIG. 1 or static X-ray imaging system 530 of FIG. 5, can include one or more repositionable imagers. In such embodiments, the repositionable images can be repositioned relative to a couch of the associated radiation therapy system, to enable a better field of view during the treatment fraction. One such embodiment is described below in conjunction with FIG. 7.

FIG. 7 is a schematic plan view of a static X-ray imaging system 730 with repositionable imagers and a couch 708, according to various embodiments. In the embodiment illustrated in FIG. 7, components of static X-ray imaging system 730 are mounted on or coupled to couch 708 of a radiation therapy system (not shown), including a first X-ray imager 731, a second X-ray imager 732, a first X-ray source 733, and a second X-ray source 734. Further, couch 708 is coupled to a couch positioning assembly 701 that is is configured to precisely position couch 708 with respect to an isocenter (not shown) of a radiation therapy system that includes couch 708.

According to various embodiments, one or more of first X-ray imager 731, second X-ray imager 732, first X-ray source 733, and/or second X-ray source 734 can be repositioned prior to a treatment fraction. In some embodiments, first X-ray imager 731 and first X-ray source 733 are adjustably coupled to couch 708, for example via a hinge, swing-arm, linkage, or other adjustable positioning mechanism. In such embodiments, first X-ray imager 731 can be repositioned to an adjusted position 751 (dashed lines) and first X-ray source 733 can be repositioned to an adjusted position 752. In this way, a bony structure, medical device, or other high-density object can be prevented from obscuring a target volume, an organ-at-risk (OAR), or other critical internal anatomical structure in X-ray images acquired by first X-ray imager 731. Thus, in such embodiments, a treatment position of a patient is not modified to enable first X-ray imager 731 to acquire suitable position verification X-ray images.

In some embodiments, one or more of first X-ray imager 731, second X-ray imager 732, first X-ray source 733, and/or second X-ray source 734 can be rotated, translated, or otherwise repositioned prior to a treatment fraction being performed on a patient. It is noted that after being repositioned and during the treatment fraction, first X-ray imager 731, second X-ray imager 732, first X-ray source 733, and second X-ray source 734 remain fixed in position relative to couch 708. Therefore, during the treatment fraction, images acquired by static X-ray imaging system 730 are taken in a patient coordinate system.

### Intra-Fractional Patient Position Verification

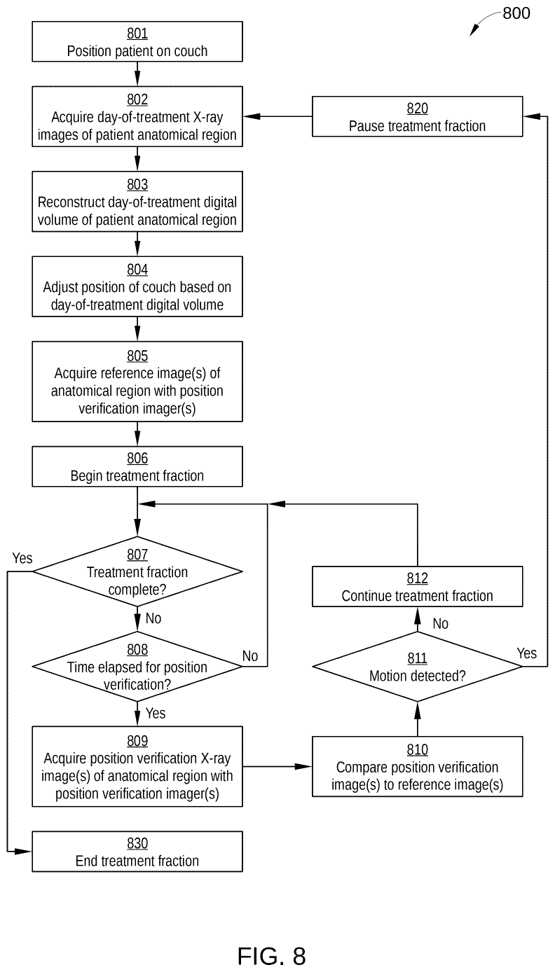

FIG. 8 is a flowchart illustrating the steps of a computer-implemented method 800 of performing a radiation therapy treatment fraction on a target volume within an anatomical region of a patient, according to various embodiments. Computer-implemented method 800 may include one or more operations, functions, or actions as illustrated by one or more of blocks 801-830. Although the blocks are illustrated in a specific order, these blocks may be performed in parallel, and/or in a different order than those described herein. Also, the various blocks may be combined into fewer blocks, divided into additional blocks, and/or eliminated based upon a specific implementation. Although the method is described in conjunction with RT system 100 of FIG. 1 and RT system 500 of FIG. 5, persons skilled in the art will understand that within the scope of the present disclosure any suitably configured system can perform computer-implemented method 800.

In some embodiments, computer-implemented method 800 is performed as part of a multi-fraction radiation therapy treatment. Generally, radiotherapy treatment fractions are employed to reduce toxic effects on healthy cells, by dividing a total dose of radiation for a patient into multiple smaller doses (fractions). Typically, each treatment fraction is delivered over a period of several or many days, for example every day for several weeks.

Computer-implemented method 800 begins at step 801, where a patient is positioned within RT system 100 for a radiotherapy treatment fraction, which can be one of multiple treatment fractions planned for the patient. In some embodiments, a patient is positioned on couch 107 so that isocenter 203 of RT system 100 is included in an anatomical region of interest of the patient. In some embodiments, tattoos or other skin markings are employed to facilitate positioning of the patient relative to RT system 100. The anatomical region the patient can be any technically feasible portion of patient anatomy that includes a PTV or other target volume 209, such as the head, chest, abdomen, and the like. In embodiments in which RT system 100 includes static X-ray imaging system 730 with repositionable imagers, one or more components of static X-ray imaging system 730 are rotated, translated, or otherwise positioned in step 801 to provide suitable X-ray images of the anatomical region.

In step 802, day-of-treatment X-ray images of the anatomical region are acquired. In some embodiments, the X-ray imaging of the anatomical region is performed in step 802 via an on-board imaging system included in RT system 100. In such embodiments, CBCT X-ray images are acquired using imaging panel 107 and imaging X-ray source 106 or by a computed tomography (CT) imaging system coupled to or associated with RT system 100. In other embodiments, magnetic resonance imaging (MRI) is performed in step 802, for example via an MRI system coupled to or associated with RT system 100.

In step 803, a day-of-treatment digital volume of the anatomical region is reconstructed based on the imaging performed in step 802. For example, in some embodiments, the day-of-treatment digital volume acquired in step 803 can be consistent with digital volume 300 in FIG. 3.

In step 804, the position of couch 107 is adjusted or shifted based on the day-of-treatment digital volume reconstructed in step 803. In some embodiments, couch 107 is shifted in step 803 so that the location of isocenter 203 corresponds with a planning isocenter of the anatomical region. Ideally, the planning isocenter is located at isocenter 203 of RT system 100 during delivery of radiation to facilitate accurate dosing of target volume 209 and to minimize or otherwise reduce dosing to OARs proximate target volume 209. In some embodiments, to determine the adjustment or shift of couch 107, the day-of-treatment digital volume is deformably registered to a planning digital volume of the anatomical region, where the planning digital volume can be a conventional CT image, a CT-quality CBCT image, or the like that is acquired prior treatment planning. The current location of target volume 209 can then be detected and a suitable adjustment or shift of couch 107 determined.

In step 805, each imager of static X-ray imaging system 130 acquires a reference image of the anatomical region. In some embodiments, the reference images are acquired immediately after the position of couch 107 has been adjusted or shifted to align the isocenter 203 to the planning isocenter of the anatomical region, for example within one second. Thus, the reference images are acquired when the patient is precisely positioned in the correct location for the treatment fraction. In some embodiments, a reference set of three-dimensional coordinate values of a particular feature disposed within the anatomical region is determined based on the reference images acquired in step 805.

In step 806, the radiotherapy treatment fraction associated with computer-implemented method 800 begins. In some embodiments, during the course of the treatment fraction, a treatment beam is directed to a target volume from multiple angles, for example via rotation of C-arm gantry 110 of RT system 100 or gantry 610 of RT system 500.

In step 807, the determination is made whether the treatment fraction is complete. If no, the treatment fraction continues and computer-implemented method 800 returns to step 807; if yes, computer-implemented method 800 proceeds to step 830 and computer-implemented method 800 ends.

In step 808, the determination is made whether a predetermined time interval has elapsed indicating that position verification is to take place. For example, in some embodiments, the predetermined time interval can be on the order of a few seconds or as short as a fraction of a second. In some embodiments, the predetermined time interval can be a time interval corresponding to rotation of C-arm gantry 110 or gantry 610 through a specified arc. If the time interval has not elapsed, computer-implemented method 800 returns to step 807; if the time interval has elapsed, computer-implemented method 800 proceeds to step 809.

In step 809, each imager of static X-ray imaging system 130 acquires a position verification X-ray image of the anatomical region. In some embodiments, each position verification X-ray image is a two-dimensional radiograph of the anatomical region. Unlike X-ray images acquired by on-board imagers of RT system 100, the position verification X-ray images acquired by static X-ray imaging system 130 are taken with the identical viewing direction relative to the anatomical region as the reference image. This is the case even when couch 107 has been rotated to a different orientation during the treatment fraction or shifted during the treatment fraction so that isocenter 203 is aligned with a different location within the anatomical region. Because the position verification X-ray images are acquired during the treatment fraction, for example while the treatment beam is being delivered to target volume 209, immediate motion detection feedback is provided to RT system 100 for the duration of the treatment fraction. In some embodiments, a position verification set of three-dimensional coordinate values of the particular feature is determined based on the position verification X-ray images acquired in step 809. As with the position verification X-ray images, the position verification set of three-dimensional coordinate values is based on imaging information acquired with the identical viewing direction (relative to the anatomical region) as the imaging information on which the reference set of three-dimensional coordinate values is based. As described below, the position verification set of three-dimensional coordinate values can be employed to detect patient and/or internal anatomy motion.

In some embodiments, to reduce the effects of X-ray scatter on the quality of the position verification X-ray images, acquisition of the position verification X-ray images in step 809 is interleaved between pulses of the treatment beam being delivered to target volume 209. For example, in some embodiments, acquisition of the position verification X-ray images in step 809 is timed to occur between two treatment beam pulses. Alternatively, in some embodiments, one or more treatment beam pules that coincide with acquisition of the position verification X-ray images are paused or skipped. In such embodiments, additional treatment beam pulses directed to the target volume can be appended to the treatment fraction as “make-up” pulses. Alternatively or additionally, in some embodiments, residual charge in the imagers of static X-ray imaging system 130 is cleared immediately before image acquisition, thereby enabling acquisition of position verification X-ray images throughout the treatment fraction.

In step 810, each position verification X-ray images acquired in step 809 is compared to a corresponding reference image. For example, in an embodiment, the position verification X-ray image acquired by first X-ray imager 131 is compared to the corresponding reference image acquired by first X-ray imager 131 in step 805, while the position verification X-ray image acquired by second X-ray imager 132 is compared to the corresponding reference image acquired by second X-ray imager 132 in step 805. Alternatively or additionally, in some embodiments, in step 810, the position verification set of three-dimensional coordinate values determined in step 809 is compared to the reference set of three-dimensional coordinate values determined in step 805.

In step 811, the determination is made whether motion has been detected based on the comparison of position verification X-ray images and reference images and/or on the comparison of the position verification set of three-dimensional coordinate values and reference set of three-dimensional coordinate values performed in step 810. For example, in some embodiments, to detect patient and/or internal anatomy motion, the position verification X-ray image acquired by an imager of static X-ray imaging system 130 is directly compared to the reference image acquired by that imager. In such embodiments, internal anatomy motion and/or motion by the patient as a whole may be indicated when a certain motion threshold is exceeded. Determining that the motion threshold is exceeded can be based on the reference X-ray image, the position verification X-ray image, and any technically feasible motion detection and/or pattern recognition algorithm. Alternatively or additionally, in some embodiments determining that the motion threshold is exceeded can be based on the reference set of three-dimensional coordinate values, the position verification set of three-dimensional coordinate values, and any technically feasible motion detection and/or pattern recognition algorithm. If motion of the patient an/or internal anatomy has been detected, computer-implemented method 800 proceeds to step 820; if no motion has been detected, computer-implemented method 800 proceeds to step 812.

In step 812, the treatment fraction continues. For example, treatment beam pulses continue to be directed to the target volume and/or C-arm gantry 110 or gantry 610 continues to rotate through a specified treatment arc at a specified rotational velocity.

In step 820, which is performed in response to motion being detected, the treatment fraction is paused. In some embodiments, delivery of the treatment beam is stopped and rotation of C-arm gantry 110 or gantry 610 is halted. In some embodiments, the patient is repositioned on and/or unloaded from RT system 100. Computer-implemented method 800 then proceeds to step 802, where a new set of day-of-treatment X-ray images are acquired and a new day-of-treatment digital volume is reconstructed. In some embodiments, upon resumption of the treatment fraction in step 806, C-arm gantry 110 or gantry 610 is positioned (for example in a reverse rotation direction from the pause location) to enable delivery of the treatment from the remaining delivery angles when the treatment fraction is resumed.

### Example Computing Device

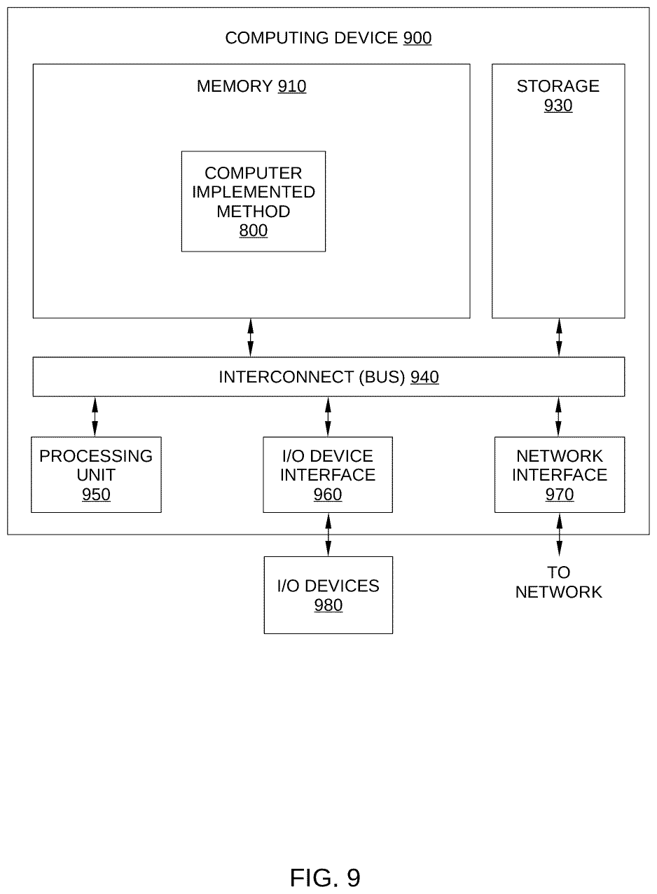

FIG. 9 is an illustration of a computing device 900 configured to perform various embodiments of the present disclosure. For example, in some embodiments, computing device 900 can be implemented as image acquisition and treatment control computer 109 of RT system 100 or image acquisition and treatment control computer 506 of RT system 500. Computing device 900 may be a desktop computer, a laptop computer, a smart phone, or any other type of computing device suitable for practicing one or more embodiments of the present disclosure. In operation, computing device 900 is configured to execute instructions associated with computer-implemented method 800, as described herein. It is noted that the computing device described herein is illustrative and that any other technically feasible configurations fall within the scope of the present disclosure.

As shown, computing device 900 includes, without limitation, an interconnect (bus) 940 that connects a processing unit 950, an input/output (I/O) device interface 960 coupled to input/output (I/O) devices 980, memory 910, a storage 930, and a network interface 970. Processing unit 950 may be any suitable processor implemented as a central processing unit (CPU), a graphics processing unit (GPU), an application-specific integrated circuit (ASIC), a field programmable gate array (FPGA), any other type of processing unit, or a combination of different processing units, such as a CPU configured to operate in conjunction with a GPU or digital signal processor (DSP). In general, processing unit 950 may be any technically feasible hardware unit capable of processing data and/or executing software applications, including computer-implemented method 800.

I/O devices 980 may include devices capable of providing input, such as a keyboard, a mouse, a touch-sensitive screen, and so forth, as well as devices capable of providing output, such as a display device and the like. Additionally, I/O devices 980 may include devices capable of both receiving input and providing output, such as a touchscreen, a universal serial bus (USB) port, and so forth. I/O devices 980 may be configured to receive various types of input from an end-user of computing device 900, and to also provide various types of output to the end-user of computing device 900, such as displayed digital images or digital videos. In some embodiments, one or more of I/O devices 980 are configured to couple computing device 900 to a network.

Memory 910 may include a random access memory (RAM) module, a flash memory unit, or any other type of memory unit or combination thereof. Processing unit 950, I/O device interface 960, and network interface 970 are configured to read data from and write data to memory 910. Memory 910 includes various software programs that can be executed by processor 950 and application data associated with said software programs, including computer-implemented method 800.

### Example Computer Program Product

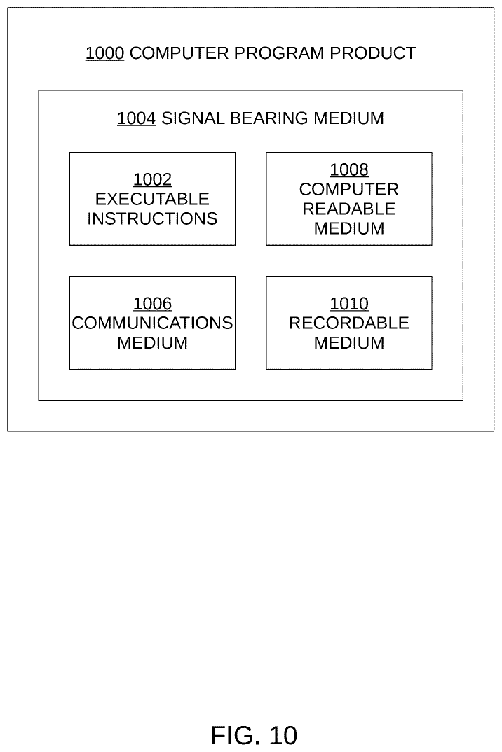

FIG. 10 is a block diagram of an illustrative embodiment of a computer program product 1000 for implementing a method for performing a radiation therapy treatment fraction on a target volume within an anatomical region of a patient, according to various embodiments. Computer program product 1000 may include a signal bearing medium 1004. Signal bearing medium 1004 may include one or more sets of executable instructions 1002 that, when executed by, for example, a processor of a computing device, may provide at least the functionality described above with respect to FIGS. 1-9.

In some implementations, signal bearing medium 1004 may encompass a non-transitory computer readable medium 1008, such as, but not limited to, a hard disk drive, a Compact Disc (CD), a Digital Video Disk (DVD), a digital tape, memory, etc. In some implementations, signal bearing medium 1004 may encompass a recordable medium 1010, such as, but not limited to, memory, read/write (R/W) CDs, R/W DVDs, etc. In some implementations, signal bearing medium 1004 may encompass a communications medium 1006, such as, but not limited to, a digital and/or an analog communication medium (e.g., a fiber optic cable, a waveguide, a wired communications link, a wireless communication link, etc.). Computer program product 1000 may be recorded on non-transitory computer readable medium 1008 or another similar recordable medium 1010.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

While various aspects and embodiments have been disclosed herein, other aspects and embodiments will be apparent to those skilled in the art without departing from the scope and spirit of the described embodiments. The various aspects and embodiments disclosed herein are for purposes of illustration and are not intended to be limiting, with the true scope and spirit being indicated by the following claims.

1. A radiation therapy system comprising:
a rotatable gantry that is configured to rotate about a treatment isocenter of the radiation therapy system;
a treatment-delivering radiation source that is coupled to the rotatable gantry and directs a treatment beam through the treatment isocenter of the radiation therapy system;
a first X-ray imager that is coupled to the rotatable gantry; and
a second X-ray imager that is mounted on a component of the radiation therapy system and is fixed in position relative to a couch of the radiation therapy system while the rotatable gantry rotates about the treatment isocenter.

2. The radiation treatment system of claim 1, wherein the component of the radiation therapy system comprises a stationary support structure on which the rotatable gantry is rotatably coupled.

3. The radiation treatment system of claim 2, wherein the support structure circumscribes a bore of the radiation therapy system.

4. The radiation treatment system of claim 3, further comprising a bore cover that is disposed between the second X-ray imager and the couch.

5. The radiation treatment system of claim 2, further comprising an imaging X-ray source for the second X-ray imager, wherein the imaging X-ray source is mounted on the stationary support structure and is fixed in position relative to the couch while the rotatable gantry rotates about the treatment isocenter.

6. The radiation treatment system of claim 2, further comprising a third X-ray imager that is mounted on the stationary support structure and is fixed in position relative to the couch while the rotatable gantry rotates about the treatment isocenter.

7. The radiation treatment system of claim 6, wherein the second X-ray imager and the third X-ray imager comprise a stereoscopic imaging system.

8. The radiation treatment system of claim 6, wherein a stereo imaging angle between the second X-ray imager and the third X-ray imager is 90 degrees.

9. The radiation treatment system of claim 1, wherein the component of the radiation therapy system comprises the couch of the radiation therapy system.

10. The radiation treatment system of claim 9, further comprising a third X-ray imager that is mounted on the couch and is fixed in position relative to the couch while the rotatable gantry rotates about the treatment isocenter.

11. The radiation treatment system of claim 9, wherein the third X-ray imager comprises a curved X-ray-receiving surface.

12. The radiation treatment system of claim 10, wherein the second X-ray imager and the third X-ray imager comprise a stereoscopic imaging system.

13. The radiation treatment system of claim 9, further comprising a couch-positioning assembly for rotating the couch about a center of rotation.

14. The radiation treatment system of claim 13, wherein the center of rotation is vertically aligned with the treatment isocenter.

15. The radiation treatment system of claim 1, wherein the second X-ray imager comprises a curved X-ray-receiving surface.

16. The radiation treatment system of claim 1, further comprising an X-ray source for the second X-ray imager, wherein the X-ray source includes a fixed anode.

17. A computer-implemented method of performing a radiation therapy treatment fraction on a target volume within an anatomical region of a patient, the method comprising:
while the patient is disposed on a couch of a radiation therapy system, acquiring multiple X-ray images of the anatomical region with a first X-ray imager that is coupled to a rotatable gantry of the radiation therapy system;
reconstructing a digital volume of the region based on the multiple X-ray images of the anatomical region;
adjusting a position of the couch based on the digital volume;
after adjusting the position of the couch, acquiring at least one reference X-ray image of the anatomical region with a second X-ray imager that is mounted on a component of the radiation therapy system and is fixed in position relative to the couch while the rotatable gantry rotates about a treatment isocenter of the radiation therapy system;
after acquiring the at least one reference X-ray image, beginning the treatment fraction;
during the treatment fraction, acquiring at least one position verification X-ray image of the anatomical region with the second X-ray imager; and
determining a motion threshold is exceeded based on the reference X-ray image and the position verification X-ray image.

18. The computer-implemented method of claim 17, wherein determining the motion threshold is exceeded comprises comparing the at least one reference X-ray image to the at least one position verification X-ray image.

19. The computer-implemented method of claim 17, wherein determining the motion threshold is exceeded comprises comparing a first set of three-dimensional coordinate values of a feature disposed within the anatomical region to a second set of three-dimensional coordinate values of the feature, wherein the first set of three-dimensional coordinate values is determined based on a first pair of images included in the at least one reference X-ray image and the second set of three-dimensional coordinate values is determined based on a second pair of images included in the at least one position verification X-ray image.

20. The computer-implemented method of claim 17, further comprising, prior to acquiring the multiple X-ray images of the anatomical region with the first X-ray imager, rotating the couch about the treatment isocenter to a treatment position.

21. The computer-implemented method of claim 17, wherein the reference X-ray image comprises a first radiograph and the position verification X-ray image comprises a second radiograph.
